(19) United States
(12) Patent Application Publication
(10) Pub. No.: US 2012/0053318 A1
(43) Pub. Date: Mar. 1, 2012

(54) METHOD OF RADIOLABELING

(75) Inventors: Ryan Simms, Mississauga (CA); Karin Stephenson, Burlington (CA); Darren Weaver, Burlington (CA); John Valliant, Ancaster (CA)

(73) Assignee: CENTRE FOR PROBE DEVELOPMENT AND COMMERCIALIZATION, Hamilton (CA)

(21) Appl. No.: 13/223,681

(22) Filed: Sep. 1, 2011

Related U.S. Application Data
(60) Provisional application No. 61/379,040, filed on Sep. 1, 2010.

(30) Foreign Application Priority Data
Sep. 1, 2010 (CA) 2714205

(51) Int. Cl. C07K 14/62; C07K 7/08; C07K 7/06

(52) U.S. Cl. 530/303; 530/328; 530/327

(57) ABSTRACT

The application describes a method of radiolabeling a molecule comprising reacting the molecule and a radionuclide labeling reagent in an emulsion, such as an oil-in-water emulsion.

### RELATED APPLICATIONS

The present application claims the benefit under 35 USC §119(e) to co-pending U.S. provisional patent application No. 61/379,040 filed on Sep. 1, 2010, the contents of which are incorporated herein by reference in their entirety.

### FIELD

The present disclosure is in the field of radiolabeling molecules and in particular relates to a new method of labeling a molecule with a radionuclide using droplet microreactors formed in emulsions.

### INTRODUCTION

Molecular imaging (MI) provides a non-invasive method to monitor biological processes in vivo which is relevant to diagnostic medicine, monitoring therapeutic response and drug development.[1, 2] Single photon emission computed tomography (SPECT),[3, 4] one of the MI modalities, utilizes compounds which seek out specific targets and that contain medical isotopes such as ¹¹¹In, ¹²³I, ⁶⁷Ga, and ^{99m}Tc to generate images from the gamma rays (γ) emitted. The γ energy of ^{99m}Tc, still the most widely used radionuclide in diagnostic medicine, is 140 keV which is close to optimal for imaging with commercial gamma cameras, and the 6-hour half-life of the radionuclide provides a reasonable timeframe for synthesis and in vivo accumulation of the imaging agent in the target tissue.

Research into new ^{99m}Tc radiopharmaceuticals has increasingly focused on developing receptor-specific MI agents derived from vectors that are biomolecules (peptides, nucleic acids, antibodies, etc.).[5, 6] Technetium radiopharmaceuticals are generally prepared using instant kits that consist of the ligand/vector to be labeled, a reducing agent, and assorted buffers and stabilizers. Pertechnetate (TcO₄⁻) from a ⁹⁹Mo/^{99m}Tc generator, is added to the instant kit producing the desired radiopharmaceutical ready to be administered to the patient. While this approach is suitable for perfusion type radiopharmaceuticals it is problematic for MI agents.

Protein-specific imaging agents generally require high effective specific activity, that is, the majority of the vector must contain the radionuclide to prevent saturating the receptor of interest with the non-radiolabeled precursor, which would dramatically reduce the image quality.[7] With existing instant kits there is no purification step so only low effective specific activity formulations are possible. For most medical isotopes, the current method to achieve high effective specific activity imaging agents is to conduct the synthesis with a large excess of the vector to be labeled and to then subsequently remove the non-radiolabeled vector using HPLC; a process that costs time and creates exposure risks. Furthermore, for biomolecules HPLC separation of the unlabeled from their labeled counterparts can be a challenge, and even if good separation is achievable, the vast majority of the biomolecules are not labeled and end up as waste. This is particularly problematic for emerging vectors such as proteins, affibodies or antibody fragments that are often costly and accessible in only minute quantities.[8, 9]

One method currently under development to try and address some of these issues is to employ microfluidic reactors. Microfluidics allows for radiolabeling reactions to be run using small quantities of precursor in a flow format where the unique reaction environment can improve reaction yields and reduce labeling times.[10-18] One of the challenges with microfluidic reactors is the total amount of a radiolabeled product that can be manufactured at one time is limited. Notwithstanding, the use of microfluidics serves as an example of how new labeling paradigms can create new opportunities to develop and translate novel molecular imaging probes.

Colloidal water droplets, (micro)emulsions and micelles have been found to accelerate the rate of some organic reactions, catalytic reactions and polymerizations[19]

### SUMMARY

A new platform for radiolabeling substrate molecules with radionuclides such as Tc(I) that significantly decreases the amount of substrate and the reaction temperature needed while maintaining or reducing the overall synthesis time, improving radiochemical purity and radiochemical yield has been developed. The platform is based on droplet reactors derived from emulsions, such as water-in-oil emulsions that are formed by mixing an aqueous phase (containing the reactants), with an oil phase (with or without emulsifying agent) where, in a model system, chelate-derived peptides (10 nmol) were labeled with [^{99m}Tc(CO)₃(OH₂)₃]⁺ in greater than 98% radiochemical yield in 10 minutes at room temperature. Conventional labeling techniques run in parallel yielded less than 3% conversion after 30 minutes for the identically formulated reaction. Using water-in-oil emulsions the amount of the peptide required to achieve reasonable labeling efficiency was further reduced to 0.5 nmol, for which the radiochemical yield was 44% after 90 minutes. The water-in-oil emulsion platform was also applied to larger molecules where, in a model system, such as novel chelate-insulin derivatives, which are a new class of probes for monitoring insulin dysregulation in vivo, the desired product was isolated in 45% radiochemical yield in 60 minutes at room temperature, which is in stark contrast to the conventional synthesis that had less than 2% yield of the desired product and for which the reaction mixture contained a significant number of impurities that precluded facile isolation of the product. Carrying out radiochemical reactions in emulsions/microemulsions is more complicated than for typical chemical transformations because of the exceeding low concentrations of the isotopes.

The present application therefore includes a method of radiolabeling a molecule comprising reacting the molecule and a radionuclide labeling reagent in an emulsion.

In an embodiment of the application the method of radiolabeling a molecule comprises:
(a) preparing a water phase by combining the molecule and the radionuclide labeling reagent in water, optionally with one or more buffering agents;
(b) combining the aqueous phase from (a) with one or more oils, and optionally one or more emulsifying agents, to form a reaction mixture;
(c) vortexing and/or sonicating the reaction mixture at a temperature of about 10° C. to about 50° C. for about 1 minute to about 100 minutes;
(d) quenching the reaction mixture; and
(e) optionally isolating the radiolabeled molecule.

Also included in the present application are kits for performing the method of the application. In an embodiment the kits comprise materials for preparing the radionuclide labeling reagent and materials for preparing an emulsion. In an embodiment the kit further includes instructions for use.

The present application also includes novel chelate insulin derivatives that are useful as tools to monitor insulin dysregulation. In particular, the derivatives are insulin that has been modified, for example, at the B1 residue with a functional group that chelates a radionuclide, for example ^{99m}Tc. In an embodiment, the functional group that chelates a radionuclide is a bis-thiazole or bis-imidazole chelating molecule that is linked to insulin molecule through a linker group, such as a short polyethylene glycol (PEG) linker, for example a PEG linker having e 1-6 ethylene oxide (EO) units, or through an alkyl amide linker. Representative examples of such compounds are compounds L3 and L4 shown in FIG. 1. The present application also includes radiolabeled versions of these insulin derivatives.

Other features and advantages of the present application will become apparent from the following detailed description. It should be understood, however, that the detailed description and the specific examples, while indicating embodiments of the application, are given by way of illustration only, since various changes and modifications within the spirit and scope of the application will become apparent to those skilled in the art from this detailed description.

### DRAWINGS

The application will now be described in greater detail with reference to the drawings in which:

FIG. 1 shows the structures of peptide ligands L1 and L2 and insulin derivatives L3 and L4.

FIG. 2 is a graph showing the percent conversion of a labeling reaction where 10 nmol of L1 peptide was labeled with [^{99m}Tc(CO)₃(OH₂)₃]⁺ in sodium acetate (0.4 mol/L) at pH 5.5 using a water in oil emulsion. SPAN80 was used as a surfactant to create varying emulsion conditions. Reactions were vortexed and sonicated according to standard procedure for 60 minutes.

FIG. 3 shows the HPLC chromatograms illustrating the low conversion of the conventional synthesis relative to the emulsion based systems. a) Conventional labeling of L1 in 0.4M Sodium Acetate at pH 5.5. b) Droplet labeling of L1 in 0.4M Sodium Acetate at pH 5.5.

FIG. 4 is a graph showing the percent conversion of a labeling reaction as a function of the volume ratio of the aqueous phase relative to the oil phase: =10 nmol L1 with no surfactant, *=1 nmol L1 with no surfactant, ▪=10 nmol L1 with 2% SPAN80 surfactant (w/w), Δ=10 nmol L1 with 5% SPAN80 surfactant (w/w). Peptides were labeled with [^{99m}Tc(CO)₃(OH₂)₃]⁺ in sodium acetate (0.4 mol/L) at pH 5.5 using a water in oil emulsion. Reactions were vortexed and sonicated according to standard procedure for 30 minutes.

FIG. 5 is a graph showing the percent conversion of a labeling reaction as a function of time: ♦=10 nmol L1, ▪=1 nmol L1, ▴=0.5 nmol L1. Peptides were labeled with [^{99m}Tc(CO)₃(OH₂)₃]⁺ in sodium acetate (0.4 mol/L) at pH 5.5 using a water in oil emulsion with no surfactant.

FIG. 6 is a graph showing the percent conversion of a labeling reaction as a function of time for SAAC-II-PEG-Insulin labeled with [^{99m}Tc(CO)₃(OH₂)₃]⁺ in sodium bicarbonate (0.5 M) at pH 8.5 using a water in oil emulsion with no surfactant. Reactions were vortexed and sonicated according to standard procedure for 60 minutes.

FIG. 7 shows HPLC traces illustrating how clean the L3 insulin labeling is in the emulsion relative to the conventional bulk labeling: a) 60 minutes bulk reaction with L3 insulin in 0.5M sodium bicarbonate. b) 60 minutes droplet reaction with L3 insulin in 0.5M sodium bicarbonate.

FIG. 8 shows HPLC traces for the L4 insulin using both conventional synthesis (a) and a water-in-oil emulsion (b). For the conventional synthesis a 100 μL solution of [^{99m}Tc(CO)₃(OH₂)₃]⁺ (555 MBq·mL⁻¹) and 100 μL of the L4-insulin (1.42 mg·mL⁻¹) in NaHCO₃ (0.1M) were added to a glass vial which was heated to 40° C. for 15 minutes. The water-in-oil emulsion used the identical aqueous phase formulation, to which 1 mL isooctane (1 wt % Span 80) was added. The emulsion was sonicated at room temperature for 15 minutes.

### DESCRIPTION OF VARIOUS EMBODIMENTS

### (i) Definitions

Unless otherwise indicated, the definitions and embodiments described in this and other sections are intended to be applicable to all embodiments and aspects of the application herein described for which they are suitable as would be understood by a person skilled in the art.

The term “agent” as used herein indicates a compound or mixture of compounds that, when added to a solution or a compound, tend to produce a particular effect on the solution's or compound's properties.

The term “emulsion” as used herein refers to a dispersion of one or more immiscible fluids in each other via the creation of an interface. In an emulsion, one liquid (the dispersed phase) is dispersed in the other (the continuous phase). Emulsions and method of fabricating emulsions are well known in the art (see for example Emulsion Science Basic Principles, 2^{nd} Edition, Leal-Calderon et al. Eds. Springer Science & Business Media, LLC, New York, 2007). The term “emulsion” as used herein includes miniemulsions and microemulsions. The term emulsions is also referred to herein as “droplet microreactors”.

The term “emulsifying agent” or “emulsifier” as used herein refers to a substance which stabilizes an emulsion, for example, by increasing its kinetic stability.

The term “pharmaceutically acceptable” as used herein means compatible with the treatment of animals, in particular humans.

The term “biomolecule compatible” as used herein means that conditions or a substance does not cause or facilitate denaturation of proteins and/or other biomolecules.

The term “biomolecule” as used herein means any of a wide variety of proteins, peptides, enzymes, antibodies and their fragments as well as other sensitive biopolymers including DNA oligomers, DNA aptamers, DNA and RNA, and complex systems including whole plant, animal and microbial cells that may be labeled using the methods described herein.

As used in this application, the singular forms “a”, “an” and “the” include plural references unless the content clearly dictates otherwise. For example, an embodiment including “an oil” should be understood to present certain aspects with one oil or two or more additional oils.

In embodiments comprising an “additional” or “second” component, the second component as used herein is chemically different from the other components or first component. A “third” component is different from the other, first, and second components, and further enumerated or “additional” components are similarly different.

The term “suitable” as used herein means that the selection of the particular conditions would depend on the specific method to be performed, but the selection would be well within the skill of a person trained in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (ii) Labeling Methods

The present application describes the use of emulsions as a new labeling platform that has significantly improved the labeling efficiency of model systems, including peptide and insulin derivatized chelates labeled with [^{99m}Tc(CO)₃(OH₂)₃]⁺, compared to conventional labeling techniques.

Accordingly, the present application includes a method of radiolabeling a molecule comprising reacting the molecule and a radionuclide labeling reagent in an emulsion.

In an embodiment of the application, the emulsion is a water-in-oil emulsion, wherein the oil is the continuous phase and water is the dispersed phase. In a further embodiment, the emulsion further comprises one or more emulsifying agents.

The oil is selected from any oil that forms a suitable emulsion with water or a water-based solution. For the radiolabeling of molecules for biological use, it is an embodiment that the oil be biomolecule-compatible. For the radiolabeling of molecules for medical use, it is an embodiment that the oil be pharmaceutically acceptable. In an embodiment of the application the oil is selected that is immiscible with water, or partially miscible with water, such that homogenization of the oil/water mixture forms an emulsion that is suitable for performing the radiolabeling reaction.

In an embodiment, the oil is selected from isooctane, mineral oil, soybean oil, silicone oils, castor oil, vegetable oil, triglycerides and perfluorocarbon liquids. In further embodiments, the oil is isooctane.

In an embodiment of the application, the ratio of the water:oil (v:v) is about 1:10 to about 1:1. In a further embodiment of the application, the ratio of the water:oil (v:v) is about 1:5.

In an embodiment, the water further includes one or more buffering agents, for example a buffering agent that adjusts the pH to about 4 to about 9. Exemplary buffering agents include, but are not limited to, sodium acetate and sodium bicarbonate. The selection of the buffering agent will depend on the solubility of the molecule and can be made by a person skilled in the art.

When present, it is an embodiment of the application that the one or more emulsifying agents are surfactants or surface active agents. Any suitable surfactant can be used in the method of the application, including, cationic, anionic, non-ionic and solid particle surfactant and mixtures thereof. It is an embodiment that the emulsifying agent is a sorbitan fatty acid, such as sorbitan monooleate (Span 80). In a further embodiment, the emulsifying agent is present in the emulsion in an amount of about 0% to about 5% by weight of the emulsion, or about 1% to about 5% by weight of the emulsion.

The radionuclide labeling reagent is any reagent that transfers a radionuclide to a target molecule. The transfer may be via any known mechanism including ionic or covalent transformations. In an embodiment, the radionuclide labeling reagent comprises a radioisotope that is useful in diagnostics and/or therapy. Examples of such radioisotopes include, but are not limited to, ^{99m}Tc, ¹⁸⁸Re, ¹⁸⁶Re, ¹¹¹In, ¹²³I, ^{, 124}I, ¹³¹I, ^{, 125}I, ⁶⁸Ga, ⁶⁴Cu, ⁶²Cu, ⁷⁶Br, ¹⁸F, ⁸⁶Y, ⁹⁰Y, ¹⁷⁷Lu, ⁸⁹Zr and ⁶⁷Ga. In a further embodiment the radionuclide labeling reagent comprises [^{99m}Tc(CO)₃]⁺, for example, [^{99m}Tc(CO)₃(OH₂)₃]⁺. The [^{99m}Tc(CO)₃]⁺ core is an attractive synthon for labeling biomolecules because it can be prepared in high yield using an instant kit and it forms stable complexes with many bifunctional chelates that are suitable for use in vivo.[20, 21] The method of the present application advantageously reduces the amount of biomolecule typically required to achieve reasonable conversion yields in the desired time frame (less than one half-life).

The molecule is any molecule that can be labeled using a radionuclide labeling reagent. In an embodiment the molecule is a biomolecule. In a further embodiment the biomolecule comprises a peptide or protein. It has advantageously been found that the method of the present application is particular amenable to the labeling of proteins, therefore it is a specific embodiment that the molecule is a protein. Radiolabeling of proteins typically requires a large excess of protein, long reaction times and/or multi-step indirect methods to drive the reaction to completion[30]. This often results in the need to use multiple purification steps which can reduce the overall yield. Using the methods of the present application, it was possible to prepare, in a single step, radiolabeled proteins using significantly lower quantities of protein. The ability to use such small quantities of protein and achieve labeling at room temperature in a reasonable time frame opens the door to labeling genetically engineered proteins that until now, because of cost and technical feasibility, would not be considered as substrates for developing radiolabel-based molecular imaging probes.

In an embodiment of the application the molar ratio of the radionuclide labeling reagent:molecule is about 1:1,000,000 to about 1:10,000, suitably about 1:100,000 to about 1:25,000, more suitably about 1:50,000. In a further embodiment, the molecule is used in an amount of about 0.5 nmol to about 50 nmol, or about 10 nmol to about 20 nmol.

In an embodiment of the application the method is performed by dissolving the molecule in the water (or buffer) and combining this aqueous solution with the radionuclide labeling reagent which is typically also comprised in an aqueous solution to obtain an aqueous phase. The oil is then added, along with the emulsifying agent(s) if used, to the aqueous phase to provide a reaction mixture. The mixture is then treated by vortexing and/or sonication at a temperature of about 10° C. to about 50° C. for about 1 minute to about 100 minutes. The reaction is then quenched using suitable means, for example, for radiolabeling with [^{99m}Tc(CO)₃(OH₂)₃]⁺, via the addition of an acid such as trifluoroacetic acid and the radiolabeled molecules isolated. In all cases the radiolabeled molecules can be isolated from the oil by simply centrifuging the emulsion.

### (iii) Kits

Also included in the present application are kits for performing the method of the application. In an embodiment the kits comprise materials for preparing the radionuclide labeling reagent and materials for preparing an emulsion. In an embodiment the kit further includes instructions for use.

In an embodiment of the application the materials for preparing the radionuclide labeling reagent include, but are not limited to, buffering agents and other agents required to prepare the radionuclide labeling reagent. For example, when the radionuclide labeling reagent is [^{99m}Tc(CO)₃(OH₂)₃]⁺, the materials for preparing the radionuclide labeling reagent may comprise sodium tartrate, sodium tetraborate decahydrate, sodium carbonate and potassium boranocarbonate.

In an embodiment of the application the materials for preparing an emulsion include, one or more oils and, optionally, one or more emulsifying agents, and related processing chemicals and devices to obtain the purified product In another embodiment, the kits further comprise one or more pharmaceutically acceptable carriers or excipients, for example for the administration of the radiolabeled molecule to a subject.

### EXAMPLES

The following Examples are set forth to aid in the understanding of the invention, and are not intended and should not be construed to limit in any way the invention set forth in the claims which follow thereafter.

### Materials and Methods

All chemicals were used as received from Sigma-Aldrich without further purification. Preparation of the peptides, synthesis of the single amino acid chelate-II (SAAC-II) and conjugation to insulin followed published methods. [23, 33]Two peptide conjugates were used in this work and two insulin derivatives: L1 (SAACII-S-G-S-G-D-Cha-F-s-r-Y-L-W-S), L2 (D-Cha-F-s-r-Y-L-W-S-SAACII), L3 (SACClI-PEG-insulig), L4 (Dithiazole-insulin) (see Scheme 1 for structures). All sonication was done in the Fisher Scientific FS20 Sonicator, and all vortexing on the Heidolph Reax Top vortex. Analytical HPLC was performed using a Waters Acquity HPLC system comprised of a Waters Acquity Binary Solvent Manager, a Waters Acquity Sample Manager, a Waters Acquity Column Manager and a Waters Acquity Photodiode Array Detector, connected to a Bioscan Flow Count radiodetector with a Waters Acquity BEH C18, 2.1×100 mm (1.7 μm) HPLC column at a flow rate of 0.25 mL·min-1 and monitoring at 254 nm and 220 nm with MBF absorbance. Column temperature was set to 35° C. and samples are cooled to 10° C. Injection volumes were set to 10 μL or 5 μL. The elution protocol used is as follows: Solvent A) H₂O containing 0.1% TFA v/v, Solvent B) acetonitrile containing 0.1% TFA v/v: Gradient elution starting at 90% A to 10% A over 9 minutes.

Caution! ^{99m}Tc is a γ-emitter (E_{γ}=140 keV, t_{1/2}=6.01 h) and should only be used in a licensed and appropriately shielded facility.

### Example 1

### Preparation of [^{99m}Tc(CO)₃(OH₂)₃]⁺

Synthesis of the [^{99m}Tc(CO)₃]⁺ core was based on a literature procedure (R. Alberto, K. Ortner, N. Wheatley, R. Schibli, A. P. Schubiger, J. Am. Chem. Soc., 123: 3135-3136 (2001); R. Alberto, R. Schibli, A. Egli, A. P. Schubiger, U. Abram, T. A. Kaden, J. Am. Chem. Soc., 120: 7987-7988 (1998)). A 2 mL Emery microwave vial containing potassium sodium tartrate (8.5 mg, 3.0×10⁻⁵ mol), sodium tetraborate decahydrate (5.4 mg, 1.4×10⁻⁵ mol), sodium carbonate (7.1 mg, 6.7×10⁻⁵ mol) and potassium boranocarbonate (4.8 mg, 3.5×10⁻⁵ mol) was crimp sealed and purged with nitrogen for 5 minutes prior to adding 2 mL [^{99m}TcO₄]⁻ (1110 MBq-30 mCi) in saline from a commercial generator. The reaction mixture was heated in the microwave at 120° C. for 7 minutes without stirring. The final solution was adjusted to pH 7 with 1.0 N HCl (190 μL). γ-HPLC: t_{R}=2.78 min; Yield: 1.11 GBq (>90% radiochemical yield).

### Example 2

### Conventional labeling of L1 and L2 peptides with [^{99m} Tc(CO)₃(OH₂)₃]⁺

A 25 μL solution of [^{99m}Tc(CO)₃(OH₂)₃]⁺ (555 MBq·mL⁻¹) and 25 μL of the L1 or L2 peptide in sodium acetate (0.4 mol/L; pH 5.5) or sodium bicarbonate (0.5 mol/L pH 8.5) were added to a vial which was left for the allotted time at room temperature. After the allotted time 25 μL trifluoroacetic acid was added to quench the reaction. A 50 μL sample was taken and added to a vial, to which 25 μL of N,N-dimethylformamide was added, and then analyzed using HPLC.

### Example 3

### Conventional labeling of L3-insulin with [^{99m}Tc(CO)₃(OH₂)₃]⁺

A 100 μL solution of [^{99m}Tc(CO)₃(OH₂)₃]⁺ (555 MBq·mL⁻¹) and 100 μL of the 0.655 mg·mL⁻¹ SAACII-PEG-insulin (0.655 mg·mL⁻¹) in sodium bicarbonate (0.5M) were added to a vial which was left for 60 minutes at room temperature. After the allotted time, 100 μL of trifluoroacetic acid was added to quench the reaction. A 50 μL sample was taken and added to a vial, to which 25 μL of N,N-dimethylformamide was added, and then analyzed using HPLC.

### Example 4

### Conventional labeling of L4-insulin with [^{99m}Tc(CO)₃(OH₂)₃]⁺

A 100 μL solution of [^{99m}Tc(CO)₃(OH₂)₃]⁺ (555 MBq·mL⁻¹) and 100 μL of the L4-insulin (1.42 mg·mL⁻¹) in sodium bicarbonate (0.1M) were added to a glass vial which was heated to 40° C. for 15 minutes. 100 μL of trifluoroacetic acid was added to quench the reaction. A 50 μL sample was taken and added to a vial, to which 25 μL of N,N-dimethylformamide was added, and then analyzed using HPLC.

### Example 5

### Labeling of the L1 and L2 peptide with [^{99m}Tc(CO)₃(OH₂)₃]⁺ using a water-in-oil emulsion

Typically, 100 μL of the L1 or L2 peptide dissolved in sodium acetate buffer (0.4 mol/L; pH 5.5) or sodium bicarbonate (0.5 mol/L; pH 8.3) and 100 μL of the [^{99m}Tc(CO)₃(OH₂)₃]⁺ (555 MBq·mL⁻¹) solution were added sequentially to a 15 mL falcon centrifuge tube. 1 mL of the oil phase (isooctane, mineral oil, soybean oil, or castor oil) containing 0, 1, 2.5 or 5 wt % Span80 was then added and the tube capped before vortexing for 3 seconds on the highest setting. The vortexed solution was then sonicated for the allotted time with re-vortexing every 5 minutes. Afterwards, trifluoroacetic acid (200 μL) was added and the solution shaken to quench the reaction. To break the emulsion, the mixture was centrifuged at 3000 rpm for 2 minutes, at which point a 50 μL aliquot of the aqueous phase and a 25 μL aliquot of N,N-dimethylformamide were added to a vial for HPLC analysis.

### Example 6

### Labeling of the L3 insulin with [^{99m}Tc(CO)₃(OH₂)₃]⁺ using a water-in-oil emulsion

Typically, 100 μL of the L3 insulin dissolved in sodium bicarbonate (0.5 mol/L; pH 8.3) and 100 μL of the [^{99m}Tc(CO)₃(OH₂)₃]⁺ (555 MBq·mL⁻¹) solution were added sequentially to a 15 mL falcon centrifuge tube, followed by 1 mL of isooctane. The vortexed solution was then sonicated for the allotted time with re-vortexing every 5 minutes. Afterwards, trifluoroacetic acid (200 μL) was added and the solution shaken to quench the reaction. To break the emulsion, the mixture was centrifuged at 3000 rpm for 2 minutes, at which point a 50 μL aliquot of the aqueous phase and a 25 μL aliquot of N,N-dimethylformamide were added to a vial for HPLC analysis.

### Example 7

### Labeling of the L4 insulin with [^{99m}Tc(CO)₃(OH₂)₃]⁺ using a water-in-oil emulsion

A 100 μL solution of [^{99m}Tc(CO)₃(OH₂)₃]⁺ (555 MBq·mL⁻¹) and 100 μL of the L4-insulin (1.42 mg·mL⁻¹) in NaHCO₃ (0.1M) were added to a 15 mL falcon centrifuge tube, followed by 1 mL of isooctane containing 1 wt % Span 80. The vortexed solution was then sonicated for 15 minutes with re-vortexing every 5 minutes. Afterwards, trifluoroacetic acid (200 μL) was added and the solution shaken to quench the reaction. To break the emulsion, the mixture was centrifuged at 3000 rpm for 2 minutes, at which point a 50 μL aliquot of the aqueous phase and a 25 μL aliquot of N,N-dimethylformamide were added to a vial for HPLC analysis.

### Results

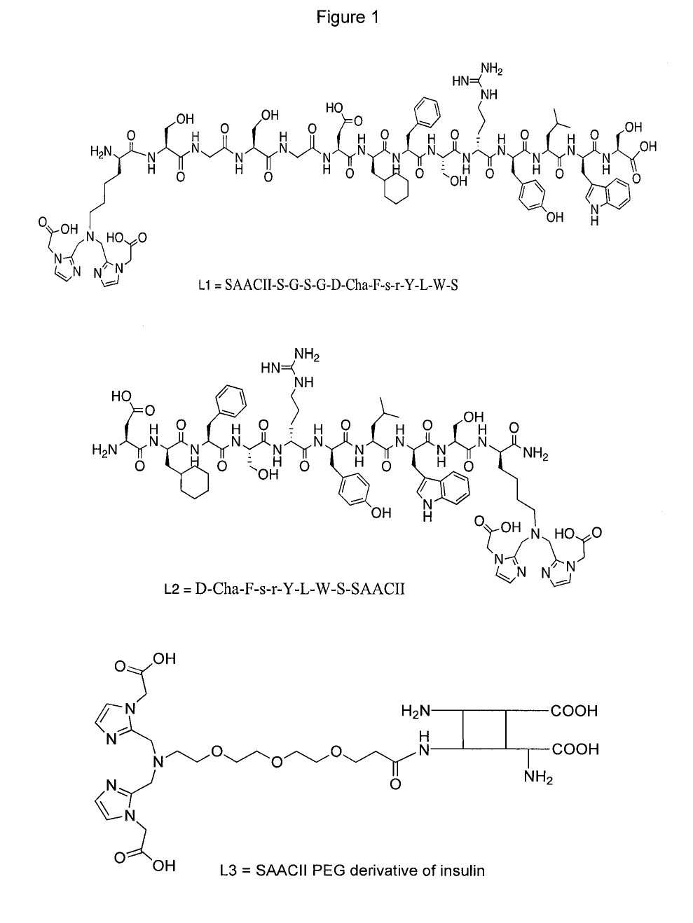

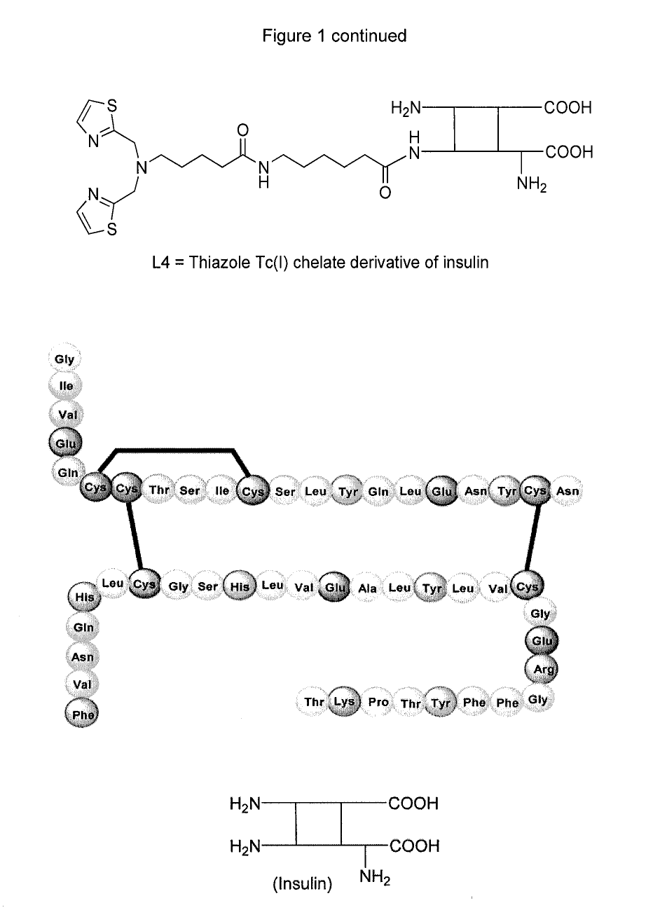

The water-in-oil emulsion platform was initially explored using two small peptides: L1 (SAACII-S-G-S-G-D-Cha-F-s-r-Y-L-W-S) and L2 (D-Cha-F-s-r-Y-L-W-S-SAACII) that are known to bind urokinase plasminogen activator receptor (uPAR) (FIG. 1). The peptides were derivatized with a tridentate ligand (single amino acid chelate-II or SAAC-II) that can be incorporated into a model peptide as if it were a natural amino acid.[23, 24] The Re complexes of the peptides, which are isostructural to the Tc analogues have low nanomolar binding affinities for uPAR (L1 Kᵢ=1.37±0.83 nM and L2 Kᵢ=0.39±0.10 nM) making them viable candidates for imaging the overexpression of uPAR which is associated with aggressive forms of cancer.[25-27]

A series of different emulsions were prepared in which the amount of surfactant, nature of the oil and surfactant, and the volume ratio between the aqueous and oil phase were varied. The labeling yields and reaction purities were compared to conventional solution phase labeling and a number of key factors including amount of peptide and reaction time were investigated. The system was derived from isooctane as the oil and sorbitan monooleate (Span 80) as the surfactant as addition of Span 80 to the water-in-oil emulsion was designed to reduce the interfacial tension that allows for smaller diameter droplets of water and formation of micelles in the oil phase.[28]

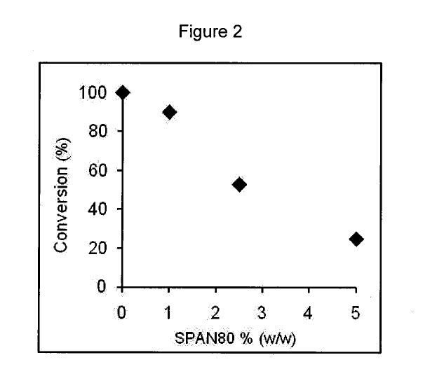

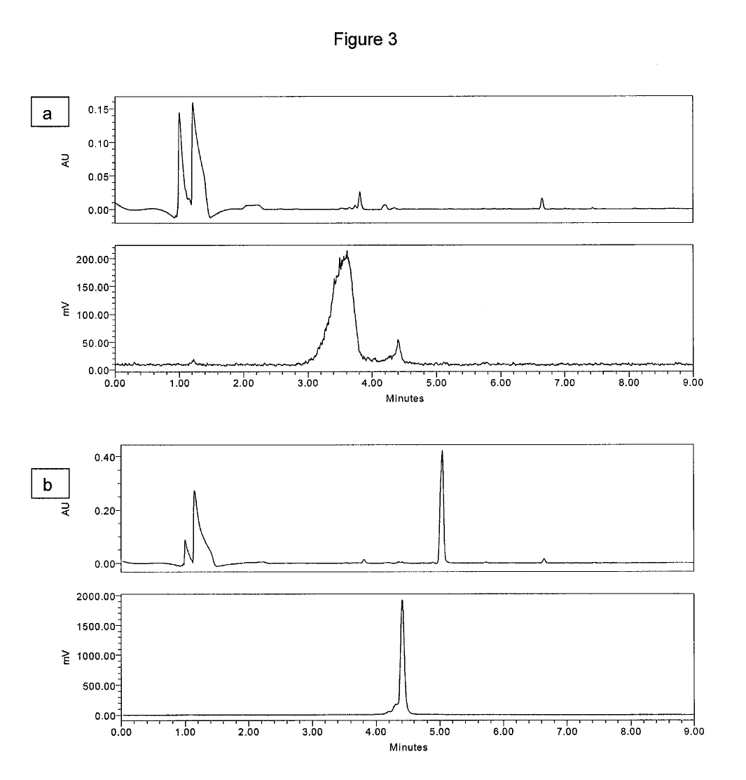

The water-in-oil emulsions investigated were prepared with a range of Span 80 in isooctane from 0-5 wt % (0-8.02×10⁻² mol/L) with 10 nmol of the peptide (L1 or L2), 37 MBq of [^{99m}Tc(CO)₃(OH₂)₃]⁺, 0.1 mL of the aqueous phase and 1.0 mL isooctane. The results for the 30 minute reaction (FIG. 2) show a clear trend that the more Span 80 added to the isooctane the lower the radiochemical yield of the labeling reaction. The optimal result was achieved when no Span 80 was added to the system and the radiochemical yield was greater than 98%. The experiment with no surfactant produces a visible emulsion that encompasses approximately 15% of the total liquid volume in the system, while the experiments that include Span 80 produce an emulsion over the entire liquid volume. Most noteworthy is that all of the labeling reactions that were conducted in emulsion outperformed the corresponding conventional labeling (i.e. the identical aqueous phase formulation with no oil added) which had a radiochemical yield of less than 3% (FIG. 3) under these conditions.

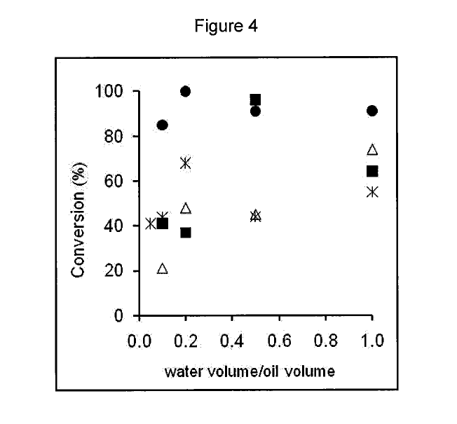

The volume ratio of the aqueous phase relative to the oil phase was also varied from 1:10 to 1:1 (FIG. 4), to determine if the aqueous:oil volume ratio affected the radiochemical yield when other relevant parameters are left unchanged. The oil volume was maintained at 1 mL with 5, 2 or 0 wt % Span 80 added to the oil phase, and the volume of the aqueous phase ranged from 0.1 mL to 1.0 mL. When the emulsion contained 5 wt % Span 80 there was an upward trend in radiochemical yield with an increased aqueous:oil ratio. With a surfactant loading of 2 wt % the relationship between the aqueous:oil ratio and the radiochemical yield of the labeling reaction became more complex. The labeling reaction was low yielding at an aqueous:oil ratio of 1:10, reaching a maximum labeling efficiency when the ratio was increased to 1:2, then dropping off when the ratio was increased further to 1:1. With no Span 80 the radiochemical yield was >98% independent of the aqueous:oil ratio. In order to further challenge whether the aqueous:oil ratio affected the yield with no Span 80 in the system the number of moles of the peptide was reduced from 10 nmol to 1 nmol. At the lower peptide concentration the aqueous:oil ratio had no discernable effect on the radiochemical yield of the labeling reaction.

Having determined that the surfactant-free emulsion provides the highest yields, the focus shifted to preparing emulsions from different oils. This included mineral oil, soybean oil, and castor oil. Oils were selected based on biocompatibility (LD₅₀ for all oils, including isooctane are greater than 2000 mg/kg) as well as their physical properties (viscosity, interfacial tension) that could influence the properties of the emulsion (formation, droplet diameter, stability, etc.). All of the oils investigated formed emulsions that outperformed conventional bulk labeling methods using a variety of reaction conditions including the type of peptide, amount of peptide, pH and buffer (Table 1). The emulsions formed with isooctane, which has the lowest viscosity at 0.5 cP, resulted in the highest radiochemical yield under all the conditions investigated. The type of oil used to form the emulsions was found to have an effect on the radiochemical yield of the labeling reaction. In general the trend showed that the yield improved as the viscosity of the oil was lowered (isooctane>mineral>soybean>castor). The use of soybean oil facilitated the oxidation of [^{99m}Tc(CO)₃]⁺ back to TcO₄⁻, which was only apparent in other cases when the labeling reaction was slow due to a low peptide concentration (1 nmol) or a buffer was used that reduced the reaction rate (Table 1).

After completing the initial optimization steps, the optimal formulation for water-in-oil emulsion was a surfactant-free emulsion with isooctane as the continuous oil phase and a volumetric aqueous:oil phase ratio of 1:5. These conditions were used to determine the minimum amount of L1 peptide that could be labeled in 30 minutes at room temperature using 37 MBq of [^{99m}Tc(CO)₃(OH₂)₃]⁺. At 10 nmol of peptide (molar ratio of L1:Tc˜50 000) the radiochemical yield was >98%, which has been previously discussed and shown to be superior to the conventional labeling method that attains only 3% of the desired product (FIG. 3). A ten fold reduction in the amount of peptide to 1 nmol results in no conversion using conventional labeling after 30 minutes compared to 37% for water-in-oil emulsions. Further reduction in the amount of peptide to 0.5 nmol for water-in-oil emulsions lowers the radiochemical yield to only 9% with no conversion observed when 0.25 nmol of the peptide is used.

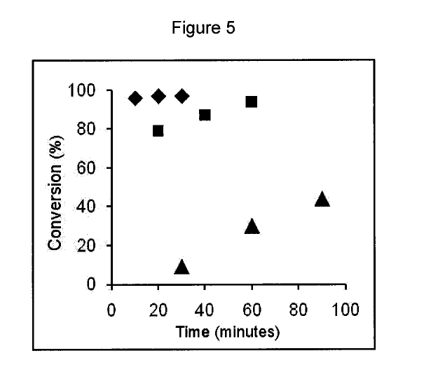

The lower quantities of vector were further evaluated as a function of time to determine if the radiochemical yield would improve if reacted for longer (FIG. 5). With a L1 peptide loading of 10 nmol the labeling reaction had a radiochemical yield>98% in less than 10 minutes. As expected, reducing the amount of the L1 peptide to 1.0 nmol resulted in slower conversion, but the water-in-oil emulsion was able to achieve a radiochemical yield of 94% in 60 minutes. At the lowest amount of peptide (0.5 nmol) the conversion increased linearly with time, achieving a yield of 44% after 90 minutes. Extending the reaction time further did not increase the yield but simply resulted in oxidation of the technetium starting material. At these quantities of L1 peptide no product was observed for conventional labeling method.

Moving beyond simple oligopeptides, two derivatives of a model protein hormone, insulin, were evaluated. The first was the SAACII chelate linked to the B1 residue of insulin by a short PEG spacer (FIG. 1, L3) and the second a thiazole derived chelate for Tc(I) also linked to the B1 site (FIG. 1, L4). The insulin derivatives are not soluble in the acetate buffer that had been used for the L1 and L2 peptides, thus a sodium bicarbonate buffer at a pH of 8.5 was used. The peptides (L1 and L2) were also run with the sodium bicarbonate buffer (Table 1) and compared to the acetate buffer (pH 5.5) showed lower radiochemical yields for identically formulated systems. However, the droplet system consistently outperformed conventional labeling methods in sodium bicarbonate at pH 8.5 which showed no conversion under the same reaction conditions.

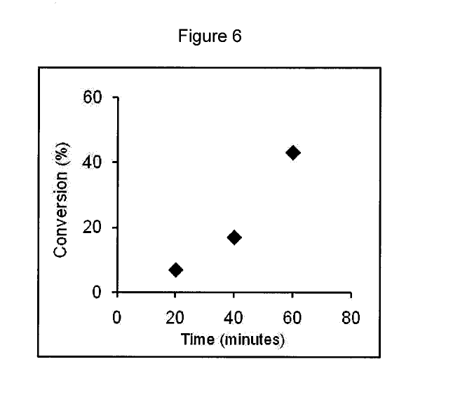

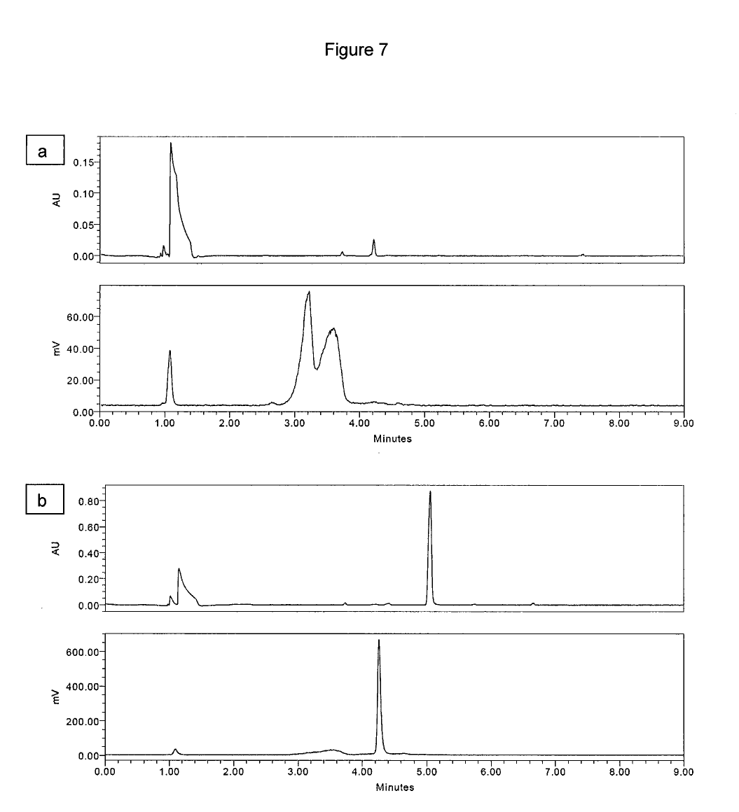

The water-in-oil labeling reaction of the first insulin derivative, L3, was evaluated with the optimized conditions described above of a surfactant-free emulsion with isooctane as the continuous oil phase and a volumetric aqueous:oil phase ratio of 1:5. The radiolabeling reaction was followed over 60 minutes for 10 nmol L3 insulin (FIG. 6) and the final radiochemical yield was 44% with no radiochemical impurities. Conversely, the identically formulated reaction using the conventional method had no discernable conversion and a large number of radiochemical impurities indicative of non-specific binding of the [^{99m}Tc(CO)₃]⁺ core to the protein derivative and/or degradation of the protein during radiolabeling (FIG. 7).

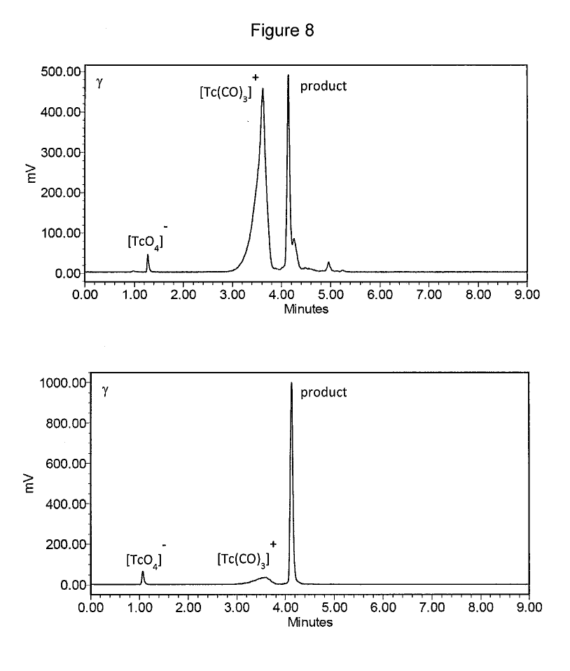

The second insulin derivative, L4, was derivatized with a thiazole derived chelate for Tc(I) linked to the same B1 site as in L3. This chelate binds in the same tridentate fashion to the [Tc(CO)₃]⁺ core and was investigated to demonstrate that the labeling technology is not specific to the SAACII ligand. The labeling of L4 insulin used a water-in-oil emulsion that contained 1 wt % Span 80 in the oil phase and was run for 15 minutes at room temperature with 85% radiochemical yield (FIG. 8) with the identically formulated conventional synthesis leading to no conversion after 15 minutes. Elevating the temperature to 40° C. in the conventional labeling produced 20% of the desired product along with some radiochemical impurities (FIG. 8) that would necessitate the use of HPLC to purify the product.

### Discussion

Here, a series of water-in-oil emulsion formulations were investigated to see if a system could be identified to optimize the labeling yields for a model system comprising vectors derivatized with chelates designed to react with [^{99m}Tc(CO)₃(OH₂)₃]⁺. This particular system was selected because of the widespread use of Tc and the fact that the [^{99m}Tc(CO)₃]⁺ core forms inert medical complexes which are ideal for designing robust in vivo probes.

The initial ligand system investigated was a peptide-based vector as there remains significant activity around developing peptide-based molecular imaging probes. Peptides can be identified that bind to virtually any protein target and their pharmacokinetic properties tuned to meet the unique requirements for in vivo imaging probes. With the SAAC system, it is now possible to radiolabel peptides at any position within the backbone since the ligand is a type of non-natural amino acid. The challenge however is that labeling often requires a large excess of the ligand to achieve good yields at room temperature in a reasonable amount of time.

Through an investigation of factors that are known to impact the properties of water-in-oil emulsions, a set of conditions were identified that could bring about quantitative labeling of extremely low concentrations of the peptide ligands. It was possible to achieve quantitative labeling in less than 30 minutes with only 10 nmol of ligand while only a nominal yield of the same product could be achieved under conventional labeling conditions. The amount of ligand could be reduced by a factor of ten if the reaction was allowed to proceed longer. Interestingly, the optimal formulation involved no surfactant which is contrary to conventional wisdom where the addition of a surfactant would reduce the water droplet diameters, increase the water-oil interfacial area and improve the colloidal stability thus typically leading to enhanced yields and reduced reaction times. Without surfactant, not only were the yields greater, the ratio of the amount of water to oil became a non-factor which greatly increases the flexibility of the system in terms of future applications.

Having seen significant improvements in radiochemical yields for peptide-based vectors, the method was applied to a larger biomolecule to evaluate the general utility of the platform. Radiolabeled proteins typically require a large excess of ligand or long reaction times or are labeled via multi-step indirect methods to drive reactions to completion.[30] This often results in the need to use multiple purification steps and can reduce the overall yield. Insulin was selected as a model system because it represents a challenging class of substrates to label and because insulin dysregulation is associated with a variety of diseases including diabetes, hypertension, and cancer[31, 32] making the parent hormone an attractive substrate from which to develop a molecular imaging probe.

Two Tc(I) chelate derived forms of insulin, whose Re complexes have been shown previously to behave like insulin in vitro, were labeled effectively with the emulsion system. While the yields were not quantitative, the only labeled compounds produced were the desired compounds and unreacted starting material and residual pertechnetate which is a simple oxidation product. The latter two products are easily removed by size exclusion chromatography. Conventional labeling reactions produced a complex mixture of radiolabeled products that were nearly inseparable. In fact the only way the target compound could be made was to use a two step labeling procedure requiring extensive HPLC purification and solvent evaporation steps which would complicate the translation of the agent for human use.

With the emulsion based labeling system, it is possible to prepare in a single step, radiolabeled analogues of insulin in a single step using 66 μg of precursor. The ability to use such small quantities of precursor and achieve labeling at room temperature in a reasonable time frame opens the door to labeling genetically engineered proteins that until now, because of cost and technical feasibility, would not be considered as substrates for developing Tc-based molecular imaging probes. In all cases the products can be isolated from the oil by simply centrifuging the emulsion at 3000 rpm for 2 minutes. Because the products can be produced in biocompatible oils, it may be possible to develop instant kits in which the emulsion containing the product is administered directly. This represents the emergence of a new class of kits that are designed specifically for the molecular imaging era.

The optimized system was a surfactant-free emulsion generated using isooctane as the continuous phase. With a peptide loading of 10 nmol the labeling reaction had a radiochemical yield>98% in less than 10 minutes, compared to no yield using conventional labeling. A reduction in the amount of peptide reduced the reaction rate, and 0.5 nmol of peptide was the lowest amount that could be efficiently labeled, achieving a yield of 44% after 90 minutes. For the insulin derivatives, water-in-oil emulsions required only 10 nmol of the vector to provide reasonable labeling efficiency with high radiochemical purity, a dramatic improvement over the conventional technique that showed <20% yield and poor radiochemical purity that would require HPLC purification.

The relevant portions of all publications, patents and patent applications are herein incorporated by reference to the same extent as if each individual publication, patent or patent application was specifically and individually indicated to be incorporated by reference. Where a term in the present application is found to be defined differently in a document incorporated herein by reference, the definition provided herein is to serve as the definition for the term.

TABLE 1
Radiolabeling results for L2-peptide sequence using different
oils, buffers and concentration of ligands.

| | Aqueous | | peptide | % | % | | |
|---|---|---|---|---|---|---|---|
| Oil | Phase | pH | (nmol) | TcO₄⁻ | Product | Viscosity | |
| Isooctane | Sodium | 5.5 | 12 | 6 | 94 | 0.50 | cP |
| | Acetate | | | | | | |
| Castor | Sodium | 5.5 | 12 | 11 | 15 | 985 | cP |
| | Acetate | | | | | | |
| Mineral | Sodium | 5.5 | 12 | 10 | 86 | 15.3 | cP |
| | Acetate | | | | | | |
| Soybean | Sodium | 5.5 | 12 | 11 | 77 | 79.1 | cP |
| | Acetate | | | | | | |
| Isooctane | Sodium | 5.5 | 1.2 | 8 | 50 | 0.50 | cP |
| | Acetate | | | | | | |
| Mineral | Sodium | 5.5 | 1.2 | 11 | 15 | 15.3 | cP |
| | Acetate | | | | | | |
| Soybean | Sodium | 5.5 | 1.2 | 28 | 7 | 79.1 | cP |
| | Acetate | | | | | | |
| Isooctane | Sodium | 8.5 | 12 | 5 | 30 | 0.50 | cP |
| | Bicarbonate | | | | | | |
| Mineral | Sodium | 8.5 | 12 | 8 | 13 | 15.3 | cP |
| | Bicarbonate | | | | | | |
| Soybean | Sodium | 8.5 | 12 | 22 | 8 | 79.1 | cP |
| | Bicarbonate | | | | | | |
| Castor | Sodium | 8.5 | 12 | 4 | 3 | 985 | cP |
| | Bicarbonate | | | | | | |
| Isooctane | Sodium | 8.5 | 12 | 4 | 20 | 0.50 | cP |
| | Bicarbonate | | | | | | |
| Mineral | Sodium | 8.5 | 12 | 11 | 7 | 15.3 | cP |
| | Bicarbonate | | | | | | |
| Soybean | Sodium | 8.5 | 12 | 27 | 5 | 79.1 | cP |
| | Bicarbonate | | | | | | |
| Castor | Sodium | 8.5 | 12 | 6 | 2 | 985 | cP |
| | Bicarbonate | | | | | | |

1. Massoud, T. F., Gambhir S. S. Genes & Dev. 17, 545-580 (2003).
2. Herschman, H. R. Science. 302, 605-608 (2003).
3. Liu, S. & Edwards, D. S. Chem. Rev. 99, 2235-2268 (1999).
4. Jurisson, S. S. & Lydon, J. D. Chem. Rev. 99, 2205-2218 (1999).
5. Liu, S. Chem. Soc. Rev. 33, 445-461 (2004).
6. Fichna, J. & Janecka, A. Bioconjugate Chem. 14, 3-17 (2002).
7. Mankoff, D. A., Link, J. M. et al. J Nucl Med. 49, 149S-163S (2008).
8. Cecilia, A., Roque, A. et al. Biotechnol. Progr. 20, 639-654 (2004).
9. de Kruif, J., Kramer, A. et. al. Hum. Antibodies. 17, 34 (2008).
10. Gillies, J. M., Prenant, C. et al. Appl. Radiat. Isot. 64, 333-336 (2006).
11. Audrain, H. Angew. Chem. Int. Ed. 46, 1772-1775 (2007).
12 Elizarov, A. M. Lab Chip. 9, 1326-1333 (2009).
13 Lu, S-Y., Watts, P. et al. Lab Chip. 4, 523-525 (2004).
14 Lee, C-C., Sui, G. et al. Science. 310, 1793-1796 (2005).
Gillies, J. M., Prenant, C. et al. Appl. Radiat. Isot. 64, 333-336 (2006).
16. Steel, C. J., O'Brien. A. T. et al. J. Label. Compd. Radiopharm. 50, 308-311 (2007).
17. Wester, H. J., Schultz, B. W. et al. Eur. J. Nucl. Med. Mol. Imaging. 36, 653-658 (2009).
18. Miller, P. W. J. Chem. Technol. Biotechnol. 84, 309-315 (2009).
19. Dwars, T., Paetzold, E. et al. Angew. Chem. Int. Ed. 44, 7174-7199 (2005).
20. Jurisson, S., Berning, D. et al. Chem. Rev. 93, 1137-1156 (2002).
21. Bartholoma, M., Valliant, J. et al. Chem. Commun. 5, 493-512 (2009).
22. Liu, Y., Liu, G. et al. Materials. 3, 3204-3217 (2010).
23. Maresca, K. P., Marquis, J. C. et al. Bioconjugate Chem. 21, 1032-1042 (2010).
24. Maresca, K. P., Hillier, S. M. et al. Bioconjugate Chem. 20, 1625-1633 (2009).
25. Lie, Z-B., Niu, G. et al. Clin Cancer Res. 14, 4758-4766 (2008).
26. Blasi, F. & Carmeliet, P. Nat. Rev. Mol. Cell. Biol. 3, 932-943 (2002).
27. Dass, K., Ahmad, A. et al. Cancer Treatment Rev. 34, 122-136 (2008).
28. Tawfik, D. S. & Griffiths, A. D. Nat. Biotech. 16, 652-656 (1998).
29. Saltiel, A. R. & Kahn, C. R. Nature. 414, 799-806 (2001).
30. Tolmachev, V. & Stone-Elander, S. Biochim Biophys. 1800, 487-510 (2010)
31. DeFronzo, R. A. Neth. J. Med. 50, 191-197 (1997).
32. Kaaks, R., Toniolo, P. et al. J. Natl. Cancer Inst. 92, 1592-1600 (2000).
33. Sundararajan, C., Besanger, T. R. et al. J. Med. Chem. 53, 2612-2621 (2010).

1. A method of radiolabeling a molecule comprising reacting the molecule and a radionuclide labeling reagent in an emulsion.

2. The method of claim 1, wherein the emulsion is a water-in-oil emulsion.

3. The method of claim 1, wherein the emulsion further comprises one or more emulsifying agents.

4. The method of claim 2, wherein the oil is biomolecule-compatible and/or pharmaceutically acceptable.

5. The method of claim 2, wherein the oil is selected that can be homogenized with water to provide an emulsion.

6. The method of claim 2, wherein the ratio of the water:oil (v:v) is about 1:10 to about 1:1, or about 1:5.

7. The method of claim 2, wherein the oil is isooctane.

8. The method of claim 2, wherein the water further includes a buffering agent.

9. The method of claim 8, wherein the buffering agent adjusts the pH to about 4 to about 9.

10. The method of claim 3, wherein the emulsifying agent is a surfactant.

11. The method of claim 10, wherein the surfactant is a cationic, anionic, non-ionic or solid particle surfactant, or a mixture thereof.

12. The method of claim 10, wherein the emulsifying agent is a sorbitan fatty acid.

13. The method of claim 3, wherein the emulsifier is present in the emulsion in an amount of about 1% to about 5% by weight of the emulsion.

14. The method of claim 1, wherein the radionuclide labeling reagent comprises a radioisotope that is useful in diagnostics and/or therapy.

15. The method of claim 14, wherein the radioisotope is ^{99m}Tc, ¹⁸⁸Re, ¹⁸⁶Re, ¹¹¹In, ¹²³I, ¹²⁴I, ¹³¹I, ¹²⁵I, ⁶⁸Ga, ⁶⁴Cu, ⁶²Cu, ⁷⁶Br, ¹⁸F, ⁸⁶Y, ⁹⁰Y, ¹⁷⁷Lu ⁸⁹Zr or ⁶⁷Ga.

16. The method of claim 15, wherein the radionuclide labeling reagent comprises [^{99m}Tc(CO)₃(OH₂)₃]⁺.

17. The method of claim 1, wherein the molecule is a biomolecule.

18. The method of claim 17, wherein the molecule comprises a peptide.

19. The method of claim 17, wherein the molecule comprises a protein.

20. The method of claim 1, wherein the molar ratio of the radionuclide labeling reagent:molecule is about 1:1,000,000 to about 1:10,000.

21. The method of claim 1, wherein the molecule is used in an amount of about 0.5 nmol to about 50 nmol, or about 10 nmol to about 20 nmol.

22. The method of claim 2 comprising
(a) preparing a water phase by combining the molecule and the radionuclide labeling reagent in water, optionally with one or more buffering agents;
(b) combining the aqueous phase from (a) with one or more oils, and optionally one or more emulsifying agents, to form a reaction mixture;
(c) vortexing and/or sonicating the reaction mixture at a temperature of about 10° C. to about 50° C. for about 1 minute to about 100 minutes;
(d) quenching the reaction mixture; and
(e) optionally isolating the radiolabeled molecule.

23. A kit for performing the method of claim 1, comprising materials for preparing the radionuclide labeling reagent and materials for preparing an emulsion.

24. An insulin derivative that comprises a functional group that chelates a radionuclide.

25. The insulin derivative of claim 24, wherein the functional group that chelates a radionuclide is attached at the B1 residue.

26. The insulin derivative of claim 24, wherein the functional group that chelates a radionuclide is a bis-thiazole or bis-imidazole chelating molecule that is linked to insulin molecule through a linker group.

27. The insulin derivative of claim 24, selected from compounds L3 and L4.

28. The insulin derivative of claim 23 further comprising a radiolabel.
